(19) United States
(12) Patent Application Publication
(10) Pub. No.: US 2024/0304281 A1
(43) Pub. Date: Sep. 12, 2024

(54) SYSTEMS AND METHODS TO GENERATE LOCAL ANCESTRY DETERMINATIONS, POLYGENIC RISK SCORES, AND OTHER USEFUL INFORMATION FROM GENOMIC DATA

(71) Applicant: Omics Edge, LLC, Middletown, DE (US)

(72) Inventors: Puya G. Yazdi, Middletown, DE (US); Deepu Unnikrishnan, Middletown, DE (US); Charles Manson Ion Lerga Jaso, Middletown, DE (US); Biljana Novkovic, Middletown, DE (US)

(21) Appl. No.: 18/597,611

(22) Filed: Mar. 6, 2024

Related U.S. Application Data
(60) Provisional application No. 63/450,395, filed on Mar. 7, 2023.

(51) Int. Cl. G16B 20/40; G16B 20/20; G16B 40/00; G16H 50/20; G16H 50/30; G16H 50/70

(52) U.S. Cl. CPC G16B 20/40; G16B 20/20; G16B 40/00; G16H 50/20; G16H 50/30; G16H 50/70

(57) ABSTRACT

The present disclosure provides methodology to generate one or more local ancestry inferences (“LAIs”) from genomic information for a target individual having a local ancestry that is at least partially unknown or unconfirmed. The LAI determinations herein can be generated using a LAI determination engine operational with a population reference database comprising a plurality of subpopulation reference datasets derived from genomic information obtained from collections of non-admixed individuals having known or determined local ancestry origins. Synthetic genomic data can also be generated to provide subpopulation reference datasets when actual genomic data having known local ancestry origin is not available. The methodology herein has utility in generation of polygenic risk scores for a target individual, among other things.

### CROSS-REFERENCE TO RELATED APPLICATIONS

This application claims priority to U.S. Provisional Application Ser. No. 63/450,395, filed Mar. 7, 2023, the disclosure of which is incorporated herein in its entirety.

### BACKGROUND OF THE DISCLOSURE

Despite vast differences in phenotypes, languages, culture, and individual preferences, any two humans living on the planet today share 99.9% of their DNA; the remaining 0.1% has been recognized to hold highly relevant information about each individual, including the part of the world from which their ancestors originated. This insight has enabled population geneticists to use DNA to infer the geographic origins of humankind. In this regard, mating between individuals in closer geographical proximity, coupled with genetic drift and divergent demographic histories, is increasingly understood today to have helped shape the modern human genetic landscape to allow any single individual to be referenced to well-differentiated reference populations via analysis of genomic information.

In addition to ancestry, an individual's genomic information can also provide clues about one's current and, increasingly, future health conditions. As to the latter, DNA can provide information about what common diseases or health-related conditions an individual may be more likely to develop as they age. By leveraging genome-wide association studies (“GWAS”), scientists are starting to uncover valuable medical insights about common polygenic diseases. These insights have, in turn, been conceptualized to allow polygenic risk scores (“PRSs”) to be derived from analysis of an individual's genomic data.

In this regard, there is growing evidence that PRSs, when accurately determined, can capture an individual's genetic predisposition to one or more diseases by generating an estimate of polygenic effects that can be associated with a likely occurrence of a disease(s) as derived from that individual's genomic data. PRSs have shown great promise for improving detection of an individual's likelihood of susceptibility to many common diseases, such as cardiometabolic diseases and certain cancers. PRSs have been particularly promising for identifying individuals at risk for early-onset disease and for improving accuracy of risk estimation in individuals carrying mutations in high-impact disease-causing genes. As the technology is further developed, experts expect that an individual's propensity to be diagnosed with a myriad of disease states at some time in their lives could give rise to new preventive care scenarios, as well as new medications and genetic treatments that could enhance clinical outcomes.

Notwithstanding the great promise of PRSs to improve diagnoses and treatments, it has been determined that the accuracy of PRSs for an individual can be highly dependent on the ancestral origin of the individuals from which the genomic data is obtained. Today, most academic and commercial investigations associated with this emerging medical technology have used genomic population datasets derived from individuals of European ancestry, such as the UK Biobank (“UKBB”). While useful for generating PRSs for individuals properly designated as having “European ancestry,” such population datasets can be less relevant in generating information for individuals of other ancestries due to, for example, differing allele frequencies and genetic architectures across populations.

By way of explanation, there are two key steps in creating risk functions for PRS: (1) calculation of weighted sums of the genetic variants using effect sizes from an independent dataset; and (2) estimation of the predictive accuracy and the dose response between the PRS and associated disease risk. Studies have repeatedly demonstrated that the predictive accuracy and/or dose response of a PRS generated for an individual having different ancestral origin profiles than that of the individuals forming the population dataset being used in the analysis does not always, and often does not, provide good correlation between individual genetic susceptibility to one or more diseases. In other words, a PRS generated from individuals having one ancestral origin profile often does not properly transfer to generation of an accurate PRS for an individual having a different ancestral origin.

There may not only be limited transferability of PRSs across ancestries, use of a PRS derived from non-aligned ancestral population data could lead to inaccurate medical information being provided for an individual having a different ancestry. For example, it has been shown that a risk of prostate cancer derivable from a population of men of “European descent” does not properly predict a risk of prostate cancer in men having ancestry generalized as being “sub-Saharan African descent,” with men from the latter category appearing to present with a significantly higher risk of becoming symptomatic at some point in their lives. It follows that a PRS generated from European-origin male individual data would likely only be marginally predictive of a non-European origin male individual's susceptibility to prostate cancer. Moreover, such a PRS provided to this individual may make it more probable that he would not see the need for active surveillance that would be indicated for someone presenting with a higher likelihood of prostate cancer incidence. In other words, this non-European-origin male could have a worse medical outcome when his genomic information is analyzed using population data that is derived from an ancestral population that is different from his own.

It may not be surprising that genomic data derived from individuals whose ancestry is derived from different continental origins (e.g., Africa vs. Europe, etc.) may not have significant reciprocal usefulness. However, ancestral differences can be seen in PRS reliability between ostensibly more discrete populations. While research into the actual or potential effects of ancestral origin on PRS generation remains in its early stages, it has become evident that many human populations that are geographically close can nonetheless be genetically heterogeneous and such regional variation can impact the genetic architecture of complex phenotypes. For example, population data derived from Northern and Southern European-origin individuals demonstrates a clear gradation in average heights across the continent, which can be attributed, at least in part, to how selective adaptation processes that, over time, shape complex traits, such as height.

Even among what would be considered to be a somewhat more “homogenous” geographic region—namely, “Northern Europe”—the accuracy of PRSs generated from models trained on one genomic dataset derived from one geographically situated subpopulation to identify the susceptibility to a disease from genomic data derived from individuals from a geographically close, but ancestrally distinct, area has been found lacking. For example, the ability to predict coronary heart disease in individuals from different “Northern European” geographic regions demonstrates varied reciprocal reliability in genomic datasets generated from the UK, Estonia, and Germany. In Africa alone, the genomic diversity and the number of ethnolinguistic groups is vast, showing extreme allele frequency divergence in many medically relevant scenarios. Similarly, genomic variability is extensive among Asians, who comprise nearly 60% of the total world population. Such genetic diversity among ostensibly closely related individuals can be translated into unequal risks for genetically associated diseases, as well as differential pharmacological effectiveness among Chinese, Indians and Malays. Lastly, despite being broadly classified as “Latinos,” the genetic makeup of admixed subjects across distinct Latin American regions can differ markedly among individuals with respect to origins from one or more of European, African, and indigenous populations. As a result, the reliability of PRSs generated for “Latinos” remains relatively marginal currently.

Given that genetics and phenotypes show significant differences and gradients across neighboring countries or even within various countries and/or geographical regions, there remains a need to develop methodologies that can generate PRSs that more precisely align with an individual's ancestral origin(s) down to a geographic area or region. There further remains a need to generate population datasets that incorporate relevant ancestral origin data for one or more populations of individuals. More broadly, there is a need to generate improved techniques to deconvolute ancestral origin information from genomic data for non-admixed and admixed individuals. The present disclosure provides these and other improvements.

### SUMMARY OF THE DISCLOSURE

Methods and/or systems for generating local ancestry inference (LAI) information for a target individual are disclosed. The methods and/or systems generate LAI information for a target individual via a LAI determination engine configured with a library of genomic information containing a collection of subpopulation reference datasets each, independently, containing genomic data derived from a plurality of non-admixed individuals. The genomic information for each of the subpopulation datasets can be generated from real-life genomic information derived from non-admixed individuals determined or known to be associated with a specific local ancestry (e.g., geographic area, region, or subregion). The population reference database provides a collection of genomic information arranged by local ancestry groupings, which can be assigned as subpopulation categories. By comparing the genomic information for the target individual with the population reference databases having local ancestry information associated therewith, LAI information for a target individual in need thereof can be generated.

The LAI determination methodology leverages a new recombination construct—a “degree of relatedness”—of a target individual to one or more subpopulations of individuals having the same or similar local ancestry. In this regard, the process can be configured to examine windows of genomic information, which can also be characterized as selected arrangements of DNA information, on a target individual's chromatids to identify a minimum number of segments that would be needed to reconstruct that window if those segments were sampled from a collection of genomic data derived from a plurality of curated reference subpopulations each having a known local ancestry. In conjunction with generation or derivation of a degree of relatedness according to the methodology herein, a data preprocessing step is conducted in which the data can be configured as windows containing contiguous subsequences of original length of DNA being evaluated. A degree of relatedness between the subject DNA and the reference sequences for each of the populations at the same positions being evaluated can be generated or derived by application of the processing method herein that is associated with the biological mechanism of recombination.

In further implementations, a population reference database configured with a plurality of subpopulation reference datasets containing genomic data generated, derived, from, or generated for, a plurality of non-admixed individuals, can be used in a determination of degree of relatedness existing between a target individual and a plurality of individuals assigned to one or more subpopulation local ancestry categories. The degree of relatedness can be derived from processing the target individual's genomic data to determine a minimum number of DNA stretches required to reconstruct a specific haplotype from a collection of non-admixed genomes in the collection of subpopulation reference datasets. A degree of relatedness of a target individual with one or more subpopulation local ancestry categories having known or determined local ancestry information associated therewith can be termed “recombination distance.” Once generated for a target individual, the generated recombination distance measurements can be processed by both convolutional and attention-based artificial intelligence layers to provide each genomic region with information associated with nearby haplotypes and global composition, respectively, to increase an accuracy of LAI generation for that target individual.

Lastly, the disclosed methods and/or systems can be configured as a two-stage pipeline: a base layer followed by a smoothing module. The inventors have recognized that the methodology herein can be aligned with the natural constraints supplied by the LAI problem. In this regard, the inventors have surprisingly determined that combination of a base layer determination of a recombination distance with a deep learning-based smoothing module synergistically leads to a new, state-of-the-art technique for accurate ancestry deconvolution for a target individual. By defining the determination of a target individual's local ancestry as a biological problem in the first order, as opposed to a mathematical problem (as in prior art approaches), the inventors have generated enhancements in ancestry deconvolution. Notably, the presented configurations for both the base layer data and smoothing layer according to the disclosure herewith has been seen by the inventors to substantially enhance retention of pertinent biological information that underlies a determination of a target individual's local ancestry, while at the same time reducing computational complexity. It is understood herein, that the preliminary degree of relatedness determined employing the base layer is also known as a first degree of relatedness. Further, the degree of related determined utilizing the smoothing layer is also known as a second degree of relatedness.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DESCRIPTION OF THE DRAWINGS

FIG. 1 is a schematic of a workflow useful in performing the methods described herein. The workflow portrayed in solid boxes and arrows denotes a more general aspect of generating local ancestry inference information (LAI) for a target individual. Incorporation of the workflow portrayed in dashed boxes and arrows represents a more specific aspect of generating LAI information for a target individual.

### DETAILED DESCRIPTION OF THE DISCLOSURE

In the following detailed description, reference is made to the accompanying drawings, which form a part hereof, and within which are shown by way of illustration, certain embodiments by which the subject matter of this disclosure may be practiced. It is to be understood that other embodiments may be utilized, and structural changes may be made without departing from the scope of the disclosure. In other words, illustrative embodiments and aspects are described below. But it will of course be appreciated that in the development of any such actual embodiment, numerous implementation-specific decisions must be made to achieve the developers' specific goals, such as compliance with system-related and business-related constraints, which may vary from one implementation to another. Moreover, it will be appreciated that such development effort may be complex and time consuming but would nevertheless be a routine undertaking for those of ordinary skill in the art having the benefit of this disclosure.

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Wherever the phrases “for example,” “such as,” “including” and the like are used herein, the phrase “and without limitation” is understood to follow unless explicitly stated otherwise.

The terms “comprising” and “including” and “involving” (and similarly “comprises” and “includes” and “involves”) are used interchangeably and mean the same thing. Specifically, each of the terms is defined consistent with the common patent law definition of “comprising” and is therefore interpreted to be an open term meaning “at least the following” and is also interpreted not to exclude additional features, limitations, aspects, etc.

The term “about” is meant to account for variations due to experimental error. All measurements or numbers are implicitly understood to be modified by the word about, even if the measurement or number is not explicitly modified by the word about.

The term “substantially” (or alternatively “effectively”) is meant to permit deviations from the descriptive term that do not negatively impact the intended purpose. Descriptive terms are implicitly understood to be modified by the word substantially, even if the term is not explicitly modified by the word “substantially.”

Estimation of ancestry is commonly known as “genetic ancestry inference,” which comprises either global or local ancestry inference. Global ancestry inference estimates the overall proportion of genomic information contributed by each ancestral population to an admixed genome. Local ancestry inference (“LAI”) estimates the number of copies of specific genomic information contributed by a particular population having the same or similar genomic information at a given site or area in respective genomes. LAI determination methodologies use the patterns of genetic variation observed at various sites along an individual's DNA to estimate an ancestral origin for various segments of an individual's DNA. Because DNA is inherited as an intact sequence with only rare, random swaps in ancestry (between the two parental DNA sequences) at each generation, ancestral SNPs form contiguous segments allowing ancestry inference based on patterns of contiguous SNP variants detectable throughout a person's genome. “Local ancestry deconvolution” refers to identifying the ancestral origin of chromosomal segments in individuals.

Broadly, the disclosure herein employs a novel methodology that allows accurate LAI information to be derived for a target individual using genomic information that is compared to a population reference database comprising a plurality of subpopulation reference datasets associated with different local ancestry information. The accurate LAI information, in turn, can be employed in a myriad of applications including, in a significant implementation, generation of one or more predictions or risk assessments for an individual in need thereof, where each generated prediction or risk assessment can be aligned with a probability that the individual has a susceptibility to one or more medical or health-related conditions associated with a population of individuals having at least some shared local ancestry with the individual. The methodology herein thus allows a target individual's genomic information to be compared with information derived from a population of individuals that are more ancestrally similar to them which, in turn, can allow enhanced accuracy when making predictions, inferences, and/or medical or health-related assessments for the first individual. Other useful information is derivable from the methodology herein, as discussed further herein.

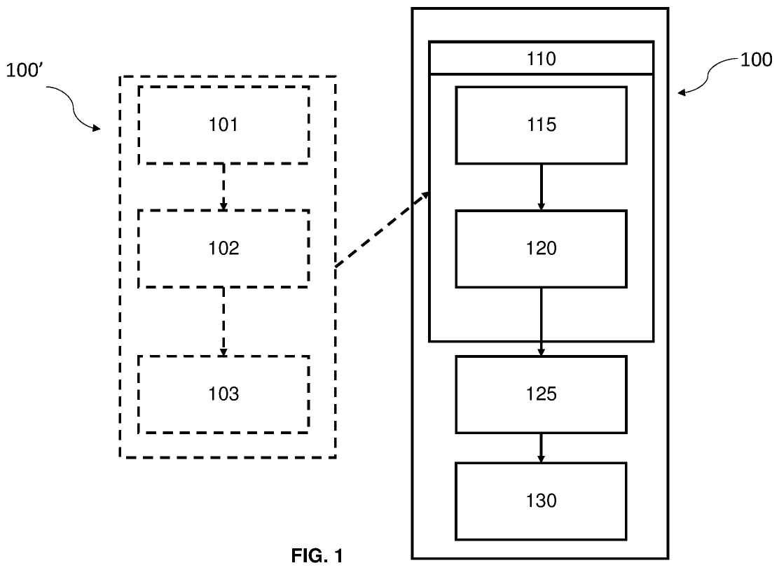

Referring to FIG. 1, process 100 provides a schematic of a methodology to generate a local ancestry inference information (LAI) for a target individual. 110 involves processing a target individual's genetic information with a LAI determination engine configured with deep learning capabilities. The process in 110 involves two aspects 115 and 120. In 115, a base layer information output from the target individual's genomic information is derived. The base layer information includes a preliminary determination of an amount of relatedness (also known as a first degree of relatedness) between the target individual and individuals having genomic information incorporated in a plurality of subpopulation reference datasets each associated with a subpopulation geographic category. Each subpopulation reference dataset contains assignments of individual LAIs for each dataset as derived from processing of genomic information obtained from collections of non-admixed individuals. Each assigned LAI is associated with a specific geographic region or location. In 120, one or more smoothing operations are performed on the base layer information output to derive a second degree of relatedness between the individuals having genomic information incorporated in each of the subpopulation reference datasets and the target individual. In 125 one or more LAI determinations for the target individual are generated on a computing device. Finally, in 130, the one or more LAI determinations are configured for use with medical or health-related information associated with the target individual.

Still referring to FIG. 1., incorporation of the workflow 100′ portrayed in dashed boxes and arrows into 100 represents a more specific aspect of generating LAI information for a target individual. In this instance, one or more aspects of the workflow 100′ are preferably performed before one or more aspects of the workflow 100. Preferably, all the aspects of 100′ are performed before the aspects of 100. In 101, a plurality of subpopulation reference datasets each associated with a subpopulation geographic category is provided. In 102, each of the subpopulation reference datasets is configured for use in a population reference database. In 103, genomic information is provided for the target individual associated with a LAI that is at least partially unknown or unconfirmed.

In an implementation, the disclosure herein provides systems and methods for generating LAI information for a target individual via a LAI determination engine configured with a library of genomic information comprising a collection of subpopulation reference datasets each, independently, comprising genomic data derived from a plurality of non-admixed individuals. The genomic information for each of the subpopulation datasets can be generated from real-life genomic information derived from non-admixed individuals determined or known to be associated with a specific local ancestry (e.g., geographic area, region, or subregion). The genomic information can also comprise synthetic genomic information generated for a collection of non-admixed individuals. The population reference database thus can provide a collection of genomic information arranged by local ancestry groupings, which can be assigned as subpopulation categories. By comparing the genomic information for the target individual with the population reference databases having local ancestry information associated therewith, LAI information for a target individual in need thereof can be generated.

In further implementations, a population reference database configured with a plurality of subpopulation reference datasets comprising genomic data generated, derived, from, or generated for, a plurality of non-admixed individuals, can be used in a determination of degree of relatedness existing between a target individual and a plurality of individuals assigned to one or more subpopulation local ancestry categories. The degree of relatedness can be derived from processing the target individual's genomic data to determine a minimum number of DNA stretches required to reconstruct a specific haplotype from a collection of non-admixed genomes in the collection of subpopulation reference datasets. A degree of relatedness of a target individual with one or more subpopulation local ancestry categories having known or determined local ancestry information associated therewith can be termed “recombination distance.” Once generated for a target individual, the generated recombination distance measurements can be processed by both convolutional and attention-based artificial intelligence layers to provide each genomic region with information associated with nearby haplotypes and global composition, respectively, so as to increase an accuracy of LAI generation for that target individual.

By way of explanation, the LAI determination methodology of the present disclosure leverages a novel recombination construct—a “degree of relatedness”—of a target individual to one or more subpopulations of individuals having the same or similar local ancestry. In this regard, the process can be configured to examine windows of genomic information, which can also be characterized as selected arrangements of DNA information, on a target individual's chromatids to identify a minimum number of segments that would be needed to reconstruct that window if those segments were sampled from a collection of genomic data derived from a plurality of curated reference subpopulations each having a known local ancestry.

By way of further explanation, in conjunction with generation or derivation of a degree of relatedness according to the methodology herein, a data preprocessing step can be conducted in which the data can be configured as windows comprising contiguous subsequences of original length of DNA being evaluated. A degree of relatedness between the subject DNA and the reference sequences for each of the populations at the same positions being evaluated can be generated or derived by application of the processing method herein that is associated with the biological mechanism of recombination.

In further implementation, the degree of relatedness can be determined based on a number of generations separating a target individual from nearest common ancestors as determined by the LAI determination engine. A smaller degree indicates a closer relationship, while a larger degree denotes a more distant connection. Here are some examples to illustrate the concept. As would be appreciated, the following categories can be assigned to various “close” relationships:
First-degree relatives: Parents and siblings share a first-degree consanguinity with their children and each other, respectively. This is because there is only one generation separating them from their nearest common ancestor.
Second-degree relatives: Grandparents, grandchildren, aunts, uncles, nephews, and nieces are all considered second-degree relatives. In this case, two generations separate them from their common ancestor.
Third-degree relatives: First cousins, great-grandparents, and great-grandchildren fall under the category of third-degree relatives. They are three generations away from their nearest common ancestor.

In various implementations, an ancestry deconvolution of a sequence of interest using a number of reference populations—that is, populations having a known ancestry—can be conducted. Using a non-limiting example for illustration, 35 reference populations having 35 known ancestries can be used in a preprocessing step to generate a 35-dimensional vector of recombination distance, with a dimension corresponding to each reference ancestry. The inventors have determined that such preprocessing can significantly reduce the dimensionality of the input data, while still retaining a relevant amount of biological data associated with the process of ancestry deconvolution for a DNA sample having unknown or unconfirmed local ancestry. In this regard, each generated dimension provides a degree of relatedness of a given sequence of sample/test DNA to a single reference ancestry population. The output of this preprocessing step can then be further processed as set out herein.

As indicated, the methodology comprises reference populations comprising known ancestries. These reference populations can comprise “curated reference subpopulations.” In an example, such curated reference subpopulations can comprise genomic information derived from a collection of non-admixed individuals having local ancestry that is well-characterized. Additional descriptions of the subpopulations reference datasets and generation thereof is discussed hereinafter. The reference subpopulations can be derived from a collection of individuals from the same ethnolinguistic group (e.g., the individuals are from a specific region and speak a particular local dialect unique to that region), village, state/province, country, and/or recognized sub-continental region (e.g., north, south, west, east, or a combination thereof, Europe/Africa/America/Asia, etc.)

The recombination distance concept utilized herein can be seen to be concordant with an assumption that shared, homologous, haplotypes are likely identical by descent. Shared haplotypes would be longer/more extensive between more closely related individuals, and shorter between and among less related individuals. Hence, two individuals with a closer more recent common ancestor would share longer segments of DNA, as compared to two individuals with more distant most recent common ancestors. The inventors thus hypothesize that a first individual having a specific local ancestry would share longer segments of DNA with individuals having that same or similar local ancestry, while they would share much shorter segments with individuals having different local ancestries. It thus follows that people having ancestors originating from the same or similar geographic locations or regions would be more likely to share more DNA together than they would with individuals having ancestors originating from other locations, especially in situations where migration was minimal or even non-existent in historical timeframes prior to the present day.

An individual's local ancestry determination can be seen as being associated with two driving forces in genetic evolution: mutation and recombination. The inventors have recognized that any data dimensionality reduction method used in determination of a target individual's local ancestry should retain these two elements of biological change while, at the same time, data should be removed, or at least de-emphasized, when it is not generally associated with the individual's local ancestry. LAI determination for a target individual can thus be improved when the processing methodology can be aligned with the underlying characteristics and context of the genomic data as it is generated in the first order. Against this recognition, the inventors have configured their LAI determination process to retain the relevant biological process-associated information, while at the same time reducing information in the analysis that can be considered to be “non-essential” or at least “less relevant” to a LAI determination.

The methodology herein can be configured as, in some implementations, a two-stage pipeline: a base layer followed by a smoothing module. The inventors have recognized that the methodology herein can be aligned with the natural constraints supplied by the LAI problem. In this regard, the inventors have surprisingly determined that combination of a base layer determination of a recombination distance with a deep learning-based smoothing module synergistically leads to a novel, state-of-the-art technique for accurate ancestry deconvolution for a target individual. By defining the determination of a target individual's local ancestry as a biological problem in the first order, as opposed to a mathematical problem (as in prior art approaches), the inventors have generated enhancements in ancestry deconvolution. Notably, the presented configurations for both the base layer data and smoothing layer according to the disclosure herewith has been seen by the inventors to substantially enhance retention of pertinent biological information that underlies a determination of a target individual's local ancestry, while at the same time reducing computational complexity.

The base layer can be configured to classify genomic windows of predetermined size by generating a recombination distance measure between a target individual's genome and genomic data derived from genomic data collections derived from or generated for non-admixed individuals having known local ancestry. This distance measure can be described as being equal to a minimum number of segments needed to reconstruct a target individual's DNA sequence of interest from the sequences present in the genomic information that makes up each reference subpopulation dataset. A generated recombination distance approximates a number of crossover events needed to reconstruct a given sequence present in the target individual's genomic information.

In a notable implementation, the recombination distance can be determined using a “greedy approach” in which a similarity matrix can be calculated by an element-to-element comparison per position and per sample, to obtain a different recombination distance between the target individual's genomic data and reference genomic data present in each group or collection of subpopulation reference datasets. The inventors have determined that a greedy approach can be effective when solving a local ancestry determination at least because a degree of relatedness can be determined by an amount of shared DNA segments between the subpopulation reference datasets and the target individual. Since a greedy approach is directed toward looking for a best option in a localized framework—here, identification of longer amounts of shared DNA where length can be assumed to be significantly determinative of shared local ancestry—the inventors have found that this processing methodology can be appropriate for use in LAI determination for a target individual.

A greedy search approach used in generating the recombination distance can allow calculations to be parallelized efficiently with the data. In this regard, the greedy search procedure can be repeated for all reference subpopulation genomic data as characterized by local ancestry to generate a vector of recombination distance for one or more genomic regions in the target individual's genomic data.

This process can be repeated for each reference subpopulation in the population reference database to generate a vector of recombination distance for the target individual's genomic data as compared to each of the reference subpopulations. The inventors have determined that the processes herein can be characterized as generating an “ancestry fingerprint” associated with a plurality of recombination distances generated from a target individual.

The generation of the described ancestry fingerprint to provide a base layer output for subsequent processing can be shown to be a significantly different approach from prior art processes that use standard machine learning techniques such as string kernels, regression, and random forests to generate probabilities associated with local ancestry determinations. The inventors herein have determined that the processing used to generate LAI information for a target individual comports with the characteristics and context by which such data is generated in nature in the first order. This, in turn, can result in more accurate LAI determinations for a target individual. In contrast to prior art methodologies that use mathematical approaches to the highly complex problem of generation of local ancestry determination, the inventors herein center their analysis according to the characteristics and context of genomic data as generated by the natural processes of recombination and mutation.

In implementations, the processes herein can be configured to examine a generated window on a target individual's chromatid to determine a minimum number of segments that would be needed to reconstruct that same window if those segments were sampled from reference subpopulation dataset genomic information as present in the population reference database, where local ancestry information associated with each subpopulation is known to be substantially accurate in the first order. This determination comprises the previously identified “recombination distance.” The LAI determination engine can be configured to commence analysis of a target individual's genomic information at a first position in that individual's chromatid, where the analysis identifies a longest continuous matching haplotype occurring in a reference subpopulation category present in a population reference database. Where a generated match stops, the process can be configured to commence again from that position to find a longest local match, and so on. In order to search efficiently for all common DNA segments between reference sequences configured with the LAI determination engine and the sample being investigated, windows of SNPs can be created for each of the target individual's chromosomes. In non-limiting implementations, this process can be carried out by using a NumPy array of the (Boolean) matches at any given position between the sample and the reference sequences (as rows) and using the product along the rows. This can allow the computation to be accelerated in a substitution of some use of memory for extra speed.

In further implementations, the processes herein can first consider a generated vector of recombination distances to be an ancestry fingerprint that can be converted nonlinearly to a measure of ancestry in terms of probabilities. In this regard, the smaller a recombination distance is to a reference subpopulation's genomic data characteristics, the higher the probability a target individual's genomic data sample came from that subpopulation, where the subpopulation can be categorized by geographic region or location. Second, by combining information from surrounding windows with the values from a subject window (i.e., a selected or identified DNA segment of interest for detection), a final local ancestry determination for the target individual can be output. The inventors have determined that the biological processes associated with a target individual's local ancestry can also make it more likely that surrounding windows are closer to each other in local ancestry than far away windows or more distant populations. Put another way, individuals who are more related in terms of local ancestry will present with more areas of similarity in their genomic data than will persons who are not as closely related in terms of local ancestry. Thus, by determining a degree of relatedness in ancestry fingerprint between a target individual and those of a collection of individuals having a known local ancestry origin—that is, a defined subpopulation of individuals—the local ancestry of the target individual can more accurately be determined.

After generation of the base layer comprising genomic identifications that provide a preliminary determination of a degree of relatedness (also known as a first degree of relatedness) between the target individual and one or more of the subpopulation categories present in the population reference database, a smoothing process can be conducted. A smoothing layer processes the provisional insights generated from the base layer to turn the vector of recombination distances of each window into a final classification using the information from the surrounding windows. Given that single-origin traces are typically longer than one window, the inventors have seen that the disclosed two-step processing can result in an increased final accuracy of the LAI determinations. In this regard, a smoothing module can be configured as a deep learning model comprising both convolutional and attention-based elements that process the base layer insights generated for each window using the information from the surrounding windows. An attention-based component has been determined by the inventors herein to be weakly associated with the global ancestry that, in practice, is reflective of real-world genomes. In this regard, a presence of a certain local ancestry can be associated with an increase in a likelihood of finding that same ancestry in other regions of a target individual's genomic information.

By way of example, the preprocessed data output that is in the form of multi-dimensional vectors can be fed into a deep learning layer as the “smoothing layer.” This smoothing layer is configured to add additional—and wider—context to each generated window in order to improve ancestry prediction for a specific window. For instance, if a majority of windows generated or derived from the base layer processing step indicate that the test/sample DNA is more likely to be closer to someone with Han Chinese ancestry it can be determined that it is more likely that the subject individual is more likely to be closely related to persons having Han Chinese ancestry. In accordance with the present methodology, the smoothing layer associated with a single window can receive the processed vectors from each of the other windows to generate a determination of local ancestry for the test/sample DNA.

The present methodology can be described as employing a combination (preferably sequential combination) of multiple CNN layers and multi-head attention layers in the smoothing layer to generate a final determination in terms of a multidimensional vector to provide a conclusion of degree of relatedness between the test/sample DNA sequence and the reference ancestry populations datasets. The inventors have determined that the disclosed approach allows use of standard techniques to generate calibrated realistic probabilities that each genomic segment is correctly associated with each of the reference ancestries in the reference population datasets.

The inventors determined that use of a small numbers of single-origin genomic models in to train the parameters enabled a limited number of latent dimensions to be derived that, in turn, retained a lesser amount of ancestry-related information. To counter this problem, the inventors herein concluded that generation of more accurate ancestry-related information retention in each layer of the deep learning smoothing layer benefits from use of a large number of samples when training the model. The greater the number of genomic information samples that can be used in training, the more ancestry-related information that can be derived from a genomic sample having fully or partially unknown or unconfirmed local ancestry information. In a non-limiting implementation, at least about 1000, at least about 5000, or at least about 7500 single-origin genome samples—that will also have known ancestry—can be used in simulation process using a genomic modeling process, such as SliM.

In notable implementations, the present methodology employs a deep learning model designed to include both convolutional and attention-based processing layers. As noted, with this approach, the inventors have determined that a weak dependence on global ancestry can be shown, since the presence of a certain local ancestry increases its likelihood of finding it in other genomic regions. The architecture of the smoothing layer therefore aligns well within the natural constraints supplied by the LAI problem.

The smoothing layer can be configured with a plurality of convolutional layers having one or more attention layers sandwiched therebetween. In a non-limiting example, the smoothing layer can be configured with five convolutional layers and two attention-based layers sandwiched between the third and fourth, and fourth and fifth convolutional layers, respectively. Other combinations of convolutional layers and attention-based layers can be used in the processing of the smoothing layer, as long as such combinations recognize the characteristics and context of the generated recombination distance that is being smoothed.

In non-limiting examples, the convolutional layers can process base layer genomic information at a chromatid window level in the case of the first two convolutional layers to bring local information associated with nearby windows in the case of the third, fourth and fifth convolutional layers. As indicated previously, the windows closest to a specific chromatid window can be inferred to be more closely associated with a person's local ancestry due to the fact that windows tend to form blocks of a given ancestry via natural recombination. Attention-based processing in combination with convolutional methodology has been found by the inventors herein to allow use of a comprehensive context to weight the base layer outputs from all other windows. This can be useful since the presence of an identified ancestry fingerprint in a target individual's genomic data even at a significant distance makes it more likely that relatedness exists between the individual and those individuals having genomic data included in one or more of the subpopulation reference datasets in the population reference database. In this regard, the attention layers can bring in information at all distances to increase information flow between each specific chromatid window, thus augmenting the local convolutional layers with global information flow and modifying their output correspondingly. Simplification of a multi-head attention layer relative to regular transformer architecture can allow use of a context long enough to span the entirety of the windowed data due to memory restrictions.

In particular, the inventors have recognized that use of an attention-based approach can reduce the complexity of analysis. Identification of specific genomic information that is of interest for detection, such as those that are known to likely be present in a subpopulation having genomic data present in the population reference database, can allow an approach herein to be suitable at least because knowledge of what one is searching in the first order for can set up the framework for LAI determination. In other words, if one knows what ancestry fingerprint elements that they are looking for, those elements can be seeded organically via training in the genomic data search parameters, thus making the disclosed approach suitable for genomic information as set out herein. Accordingly, identification of local ancestry by searching for genomic information that has a higher probability of being present in a specific subpopulation can not only simplify analysis but can also provide improvements in LAI determination accuracy.

In contrast to prior art processes, the use of convolutional and attention-based layers as disclosed herein also can provide enhanced accuracy in LAI determination. The disclosed smoothing layer attention layer pulls data from multiple parts of the entire genome for the target individual to make local calls. While the accuracy of the methodology can be expected to decrease with increasing number of generations as a result of shorter segments being inherited by the target individual, even with deeper generations—or “less relatedness”—the presently disclosed methodology can still provide enhanced LAI determination accuracy versus prior art methodologies. The architecture of the methodology thus provides a novel approach to the LAI determination problem that is in alignment with the nature of the data being analyzed and the results that are sought to be attained therefrom.

In the processes herein, the convolutional layers can be configured as moving filters that can generate different insights from the base layer output and retain generated information in parallel. This is in contrast to a normal convolutional neural network in which pooling (down sampling) layers in between the convolutional layers would be performed. In the present methodology, the information generated from the convolutional layer processing is retained and the attention-based layers are used in between to process the result of the parallel filters as a (similarity) vector space. The convolutional output thus replaces the typically generated embedding into an n-dimensional vector space in a transformer architecture to provide global information flow using proximity in this atypical (convolutional) vector space. A final convolutional layer can provide an output in terms of probabilities associated with a LAI determination. This in contrast to the fully connected layers that would typically be found in a convolutional neural network. The subpopulation from the population reference database that can be assigned a maximum probability as being associated with the target individual's genomic information is thus identified as the LAI for a target individual.

Methodology for processing a target individual's genomic information against the information in the population reference database to generate LAI information therefore can incorporate a novel process found by the inventors herein to generate a heretofore unrealized accuracy in generated LAI information for target individuals. The present disclosure can thus expand the ability to generate accurate LAI determinations even when expansive databases of genomic information for specific subpopulation local ancestry categories may not be available. Yet further, the methodology herein can allow accurate LAI information to be provided for individuals having more than one local ancestry origin, for example, for admixed individuals. Still further, LAI determinations made from conventional LAI determination methodologies can be validated using the methodology herein.

In contrast to methodologies that seek to align a target individual's risk of presenting with or acquiring a medical or health-related condition by detecting close relatives and analyzing genomics and health information associated therewith to make affirmative disease risk predictions therefrom—for example, “cousin level” relatives as in US Patent Publication No. 20210082578, the disclosure of which is incorporated herein in its entirety—the present methodology recognizes that a target individual's overall current and future medical or health-relation condition states can be associated, at least in part, with an ancestral origin. Such geographic origin information can, of course, be associated with persons who are related, as shown by genomic data; however, LAI determination expands a definition of “relatedness” to geographic location origin for ancestors. This is a distinction with a difference: because different geographic regions historically were likely subjected to different climate, food types, food amounts, etc., it can be expected that human genomic information that can be traced to ancestral origin location could be relevant to a person's susceptibility—or lack thereof—to a particular medical or health-related condition. For example, immune system characteristics have been linked to evolutionary background and have been shown to vary. People of African ancestry generally show stronger immune responses and a stronger inflammatory response compared to Europeans, which can limit the growth of bacteria. The differences in immune system activity appear to influence both internal and external microbiomes in terms of diversity and community structure which, in turn, can be understood to directly or indirectly affect an individual's current or future medical or health-related condition state(s).

Moreover, LAI determination goes beyond the characterization of someone by “ethnicity,” at least because people who ostensibly present with the same “ethnic category” may nonetheless have different local ancestries that can affect their susceptibility to one or more medical or health-related conditions. In this regard, recent studies indicate that individuals who may generally be characterized as “East Asian,” while sharing many similarities in appearance, language and culture etc., have complex genetic relationships that appear to be associated with regional isolation, initial population divergence, geographical isolation, subsequent gene flow and possibly regional natural selection. Other studies have shown local ancestral origin differences in persons classified in the ethnic category of “Hispanics,” largely due to continental ancestry patterns, has been seen to impart this category with considerable genomic diversity. It follows that such ancestral differences can be expected to affect an individual's susceptibility to certain disease states.

As would be appreciated, the accuracy of local ancestry assignments can be highly dependent on the quality of the reference panel of genomic information that is employed in the determination. That is, if the reference population data used in generating a LAI for an individual is not correct in the first order, it can be expected that LAI information generated therefrom will also not be accurate. In other words, it will be a “garbage in, garbage out” problem. Thus, the inventors herein have endeavored to generate local ancestry information for a plurality of subpopulation reference datasets that is substantially accurate before incorporation thereof in the population reference database that is used in the LAI determination engine. The manner in which this is conducted according to the disclosure herein provides notable benefits over prior art methodology.

In one aspect, the methodology herein provides a process for configuring genomic data for use in a LAI determination engine that significantly reduces the dimensionality of the data while still allowing the data to retain the substance of the biological information as needed for use in a deep learning process. The inventors have determined that, in the absence of this step, it would be very difficult to solve the problem without also having access to millions of data points for each subpopulation. While the amount of genomic information may eventually be generated for some subpopulations where there may be a large number of individuals from which data can be derived (e.g., Europeans, Chinese, South Asians, etc.), for some subpopulations, genomic data may be difficult to obtain and/or may not exist in large enough amounts to generate accurate LAI. Moreover, as discussed previously, there can be significant differences between individuals who would otherwise be determined to be “locally related” using prior art LAI determination processes. By enabling subpopulation reference datasets having smaller numbers of individual genomic data to suitably be used to identify additional, and perhaps more subtle, local ancestry differences and similarities between individuals, the present methodology can generate improvements over current methods of determining local ancestry information for target individuals. Indeed, it could be possible to extract highly nuanced differentiations in local ancestral origins using the methodologies herein even when limited data may be available for some subpopulations.

In this regard, the processes herein can allow accurate LAI determination for a target individual even when the amount of genomic data existing for a specific subpopulation to which the individual may be associated might be somewhat limited. In other words, the processes herein can enhance the determination of LAI(s) for target individuals even when it may be difficult or even impossible to generate large datasets having local ancestry accuracy for specific subpopulations associated with geographic areas or regions of the world. The inventors' processing methodology can allow input genomic data to retain substantially all the biological information relevant to generation of LAI information, while at the same time making the dimensionality of the input data smaller. This can reduce the amount of subpopulation genomic data needed for training in comparison to prior art LAI determination methodologies substantially without sacrificing the accuracy of an LAI determination for a target individual.

As discussed previously, a target individual's genomic data processed according to the two-stage process hereinabove can be analyzed via a LAI determination engine configured with a population reference database. In implementations, a population reference database can comprise a collection of genomic information generated from a collection of single-origin individuals, who can also be termed “non-admixed individuals,” where each collection of non-admixed individuals can be termed a “subpopulation reference dataset,” as referred to hereinabove. Such subpopulation reference dataset can be derived (in the case of real-life genomic data sources) or generated for (in the case of synthetic genomic data sources) for use as discussed hereinafter.

As an example of an actual data source for use in the methodology herein, genomic information derived from the UK Biobank (“UKBB”) has been found to have utility herein. The vast majority of the UK Biobank (UKBB) participants have British and Irish ancestors; however, this dataset also includes a source of much diverse ancestries beyond those having recent ancestral UK origins. In this regard, a collection of genomic data used in the LAI determination engine can be derived from non-UK origin ancestries present in the UKBB database currently, and that are likely to be present in larger volumes in the future as more data become available. The inventors have recognized that with migratory distances being much shorter a few decades ago, tracing ancestral origins by birthplace and self-reported ethnicity as tagged in the UKBB data can be used as a proxy for or to approximate local ancestry for a significant number of the individuals having genomic data incorporated therein. For example, with the influx of immigrants into the UK from the former British colonial regions in recent decades, the inventors have recognized that the UKBB can be mined for information that includes highly reliable self-reported geographic-level ancestry information. To this end, a person whose parents or grandparents arrived in the UK from an ex-UK location in the last about 20, 30, 40, 50, 60, or 70 years—a time period that can be associated with a significant amount of immigration into the UK from former British colonial regions—could be expected to provide reliable information associated with their ancestry, at least as such self-reported ancestry information can be relevant back through a reasonably limited number of generations. Of course, such self-reported information may incorporate inaccuracies for various reasons; accordingly, one or more data filtering methodologies can be used to clean the data, as discussed further hereinafter.

Additional publicly available genomic datasets as existing today can also be used in the population reference database. Such datasets can be generated from academic, government, or other sources. Examples of datasets existing today that may have utility in the present methodology are included hereinafter as Appendix I. Again, data filtering methodology can have utility in the use of disparately generated data collectively as elements of the population reference database configured for use in the LAI determination engine. To the extent applicable, the referenced articles from which the datasets are derived are incorporated herein in their entireties by this reference. Appendix II provides further clarification of the nature and content of the subpopulation reference datasets.

To this end, when different datasets generated from different protocols and different source data are combined for use in the LAI determination engine, one or more data filtering steps can be conducted. Such filtering steps can ensure that a plurality of differently-generated datasets can be properly analyzed together as a single/merged dataset—that is, in the form of a population reference database. Application of the one or more filtering steps can facilitate generation of the population reference database for use in the LAI determination engine that have a high likelihood of including subpopulation datasets that include genomic information that is correctly categorized or grouped according to the relevant subpopulations so as to allow accurate LAI information to be generated for a target individual.

For example, use of genomics data that is produced by different methodologies without first subjecting the respective datasets to any necessary transformations could embed biases or inaccuracies into any LAIs derived from a single/merged genomics dataset that is provided for use as the population reference database. To allow such differently generated datasets to be combined for use in a population reference database, genomics data derived from different sources can be subjected to a data transformation step in the first order, if necessary. As would be appreciated, a data transformation step can convert differently configured and/or formatted data into a form to allow relevant co-processing thereof.

Prior to performing a data transformation step, the need for such a step can be determined by analysis of the subject dataset. The type of data transformation suitable for a specific dataset can also be determined. In some implementations, a data transformation step may not be needed, for example, if the configuration and/or formatting of data derived from a plurality of different datasets allows processing thereof in native form.

Even for data that is determined to be appropriately configured/formatted so that it can be processed in native form, a population reference database generated from one or more genomics datasets may comprise errors/inaccuracies. Thus, data cleaning prior to use of the data in the population reference database to generate a LAI for a target individual can thus be highly beneficial, if not required.

In regard to combination of disparate datasets as a single/merged collection of genomic data as a population reference database, the individual datasets may incorporate categorizations or groupings that are not primarily associated with the actual or real local ancestry data values but that can effectively be artifacts of the subject data acquisition and/or processing steps. As an example of a filtering step that may be appropriate for the subject datasets prior to use in the population reference database, it has been determined by the inventors herein that genomic data (e.g., a specific SNP, etc.) identified in a subject dataset as being associated with a grouping or categorization of a local ancestry may actually be an artifact of the manner in which the data was processed. The data analysis protocols used to generate a subject genomics dataset may be conducted to provide “bespoke” results that are not easily transferable to more generalized inquiries, such as that actually or potentially relevant to a LAI determination for a target individual using the disclosed methodology. Such artifacts should be filtered out individually and/or the dataset can be processed to “clean” the data prior to use of the population reference database in the LAI determination engine.

In some implementations, a first filtering step can be conducted that ranks whether genomic information identified in a subject subpopulation dataset to be used in the population reference database ranks in the top end or lower end of being likely due to algorithmic or other source-dependent effects. This filtering process can substantially eliminate any information in the dataset that might, in fact, may not be associated with real or actual information relevant to determination of LAI for a target individual when the dataset is included in the population reference database. While this filtering method can remove real or accurate subpopulation genomic data from the population reference database, the inventors have determined that prophylactic elimination of data that has a higher probability of being incorrect can enhance the overall accuracy of the information deployable from a subpopulation reference dataset for use in generating a LAI for a target individual. In other words, less data may in fact generate better results in some cases. As discussed previously, the novel processing techniques generated by the inventors can allow smaller amounts of subpopulation data to be used to generate accurate LAI for a target individual. Thus, unlike prior art methods wherein a goal might be to collect as much genomic information as possible for use in a LAI determination, the present methodology can enhance subpopulation reference dataset accuracy for use in deriving LAI information for a target individual even though to do so may reduce the amount of genomic data present in the subject dataset in the first order.

Population reference database accuracy can also be enhanced by use of an additional filtering step on the population reference database to clean the data to remove that data which appears to be inaccurate. For example, a second filtering step can be conducted on the data in a subject dataset that remains after the first filtering step to remove samples in each of the subject datasets comprising genomic information, for example SNP determinations, that show disagreement between reported ancestry and genetic origin. As noted previously, while it is expected that persons having direct information about their immediate ancestors (e.g., parents, grandparents, great-grandparents) local origin, it could be expected that some persons may intentionally or unintentionally not provide accurate information thereof. Accordingly, it can be highly beneficial to review the local ancestry information as-provided with a specific dataset with other available information to remove likely or probably inaccurate information before use in the population reference database.

For example, an individual's genomic data present in the reference panel may indicate that the person is of X ancestry, but the actual genomic data for that individual could include one or more SNPs or grouping of SNPs that are not known to be associated with that person's local ancestry as reported or found in prior genomic analyses. In such a situation, a person's reported ancestry as recorded in a subject dataset may not be concordant with that person's actual genomic data as evident from the data.

Whatever the reason for a lack of alignment of the reported ancestry for an individual whose genomic data is being incorporated in the population reference database, a second filtering step can assist in making sure that whatever data is included as reference data has a high likelihood of being correct. Various methods of “cleaning up” the genomic data prior to use thereof in data analyses can be used herein.

For example, an analysis can be conducted to confirm (or disconfirm) that the SNP is correct for that ancestry. Alternatively, such questionable data can be removed from the subject dataset.

As would be appreciated by one of ordinary skill in the art, the appropriate data filtering process can be determined from an analysis of the subject genomic information datasets. Moreover, as data science continues to advance at a rapid rate, additional methodologies appropriate to process complex and multi-dimensional datasets, such as is the case with genomic data, can be expected to be developed. Such newly developed methodologies may impart improvements that can be used to generate the population reference database having utility in the methodology herein.

As a non-limiting example of a useful filtering technique developed by the inventors herein that can allow inaccurate data to be identified and eliminated prior to use in a population reference database, principal component analysis (“PCA”) can be conducted on some or all of the individual datasets prior to use thereof in the population reference database for use in the LAI determination engine. From PCA, the inventors have observed that a landscape of gradients can reveal a linking of geographically close regions. In another exemplary and non-limiting filtering process, PCA can be followed by uniform manifold approximation and projection (UMAP) along with application of t-distributed stochastic neighbor embedding (t-SNE) in genealogical nearest neighbors (GNN) statistic estimated with tsinfer. From this second mentioned filtered process, the inventors have observed that each subpopulation can tend to form a group of samples in a two-dimensional projected space, which can indicate that the closest individuals in a dataset are likely to be more genetically similar. This, in turn, could reveal outlier samples from individuals in a dataset that are not actually closely related to the group to which they supposedly should belong. Through an automated clustering process and visual inspection (i.e., at least some human supervision) of the population datasets, suspicious candidates and outliers can be eliminated, in various implementations.

An output of the one or more filtering steps can be a population reference database comprising a plurality of subpopulation reference datasets each including genomic data associated with a plurality of non-admixed individuals having local ancestries categorized or grouped from a plurality of world regions. Such local ancestry information can be designated “geographic subpopulation categories” in that the local origins for individuals in such data collections are associated with specific geographic locations or regions in the world. The generated population reference database provides local ancestry information that has utility in the LAI determination engine. In an example, the population reference database having utility in a LAI determination engine can comprise samples of genomic data from at least about 1,000, 5000, 10,000 or more non-admixed individuals, where a plurality of such individuals are each categorized or grouped in sub-populations as being associated with a local ancestry associated with a geographic region of the world. The number of non-admixed individuals having genomic information incorporated in each subpopulation reference dataset that is itself incorporated in the population reference database used in a LAI determination can be at least about 10, 15, 20, 25, 30, 35, 40, 50, 60, 70, 80, 90, 100, 200 or more. As would be appreciated, it can be beneficial to include more non-admixed individual genomic information in each of the subpopulation reference datasets, but an increase in number of individual data elements should not be elevated in importance over a significant preference for use of genomic information having local ancestry information that is known to be substantially accurate. Again, and as discussed previously, the methodology herein can provide accurate LAI determination for a target individual even when a relatively small number of individual data points for non-admixed individuals is included in a subpopulation reference dataset.

One or more data filtering steps that can enhance the accuracy of the subpopulation reference datasets and use thereof to generate one or more LAIs for a target individual can further improve operation of the LAI determination engine, such as by use in training sets associated therewith. In this regard, accurate LAI determinations can allow continuous improvement for subsequent use of the LAI determination engine. Accordingly, it can be beneficial to regularly validate the output of the LAI determination engine for a target individual. Once such output is validated to confirm the accuracy thereof, such generated information can be added back into a relevant subpopulation reference dataset to increase the data content thereof. As would be appreciated, any data added back to a subpopulation reference dataset can be coded/tagged etc. to differentiate such data from other data natively present in the subject dataset.

For some subpopulations of individuals that may be of interest for determination of local ancestry, data sources suitable for generating a LAI determination for a target individual may not exist or may not exist to allow accurate determination of local ancestry information for an individual having a specific local ancestry. For example, genomic information for some geographic region subpopulations may not be included in publicly available genomic information databases because such data has not been collected previously or has not been collected in enough quantity or quality to allow creation of reference data for that subpopulation.

In some implementations, synthetic genomic data can be used in the population reference database. Synthetic data can be described as artificially annotated information that is generated by computer algorithms or simulations. Synthetic genomics data can assist in analysis when validated, real world, genomics data is sparse, lacking, or even non-existent. A class of machine learning methods called “generative models” are increasingly being proposed to generate high quality artificial genomes for use in genomics information analysis.

As mentioned previously, due to a current research bias toward studying populations of European ancestry, many non-admixed subpopulations today are under-represented in genomic databases, which can limit the ability to create datasets for use in the methodology herein. Moreover, in order to accurately determine local origins for as many target individuals as practicable, a wide assortment of genomics data associated with disparate local/geographic origins will be needed to be present in a subpopulation reference dataset that is used in the population reference database. As discussed previously, the methodology herein can enable use of more subpopulation datasets than are currently used in prior art processes that approach LAI determination from a framework of a “math problem.” Nonetheless, at least some accurate local ancestry data needs to be present in the population reference database to allow a target individual's ancestral origins to be accurately derived for a target individual. In the present disclosure, synthetic data can be used as a substitute when suitable real-world subpopulation local ancestry data is not available.

A majority of the data held by government institutions and private companies is considered sensitive and/or proprietary and, as such, some datasets may not be easily accessible to use in the methodology herein. Synthetic data can also assist in providing or, in some implementations, serving as a proxy for real-world data when patient privacy concerns may prevent or limit the availability of genomics data for third party use. Synthetic data can be used to address the significant privacy concerns that can arise when working with genomic data, enabling faster sharing of data and enabling innovation among different research groups. In this regard, synthetic versions of real-world genomics data can be safely shared and mined for insights, without revealing any sensitive or personally identifiable information. This shareable quality can enable, for example, medical research between hospitals where a researcher can learn about a disease or diagnosis, but not the real patients that the data was based on.

In implementations, synthetic data can be used to create artificial versions of subpopulation reference datasets for use in the population reference database. Yet further, in generating and/or updating the population reference database used in the LAI determination engine, an analysis of the population reference database can be conducted to determine whether one or more subpopulations associated with individual local origins—that is, geographic regions, areas, or locations—are present or absent from the dataset. If a local origin of interest is present in the population dataset, an analysis can be conducted to provide information about the quantity and/or quality of the data incorporated in such subpopulation dataset and, if appropriate, synthetic genomics data can be generated to augment sparse or limited amounts of data, as well as to substitute for local origin subpopulation data that is determined to not be of a quality that can allow accurate LAI information to be derived therefrom. In some implementations, a subpopulation reference dataset that may be restricted from use due to privacy concerns and/or issues associated with proprietary data can be used to generate a suitable synthetic dataset for one or more subpopulations associated with a local origin of interest.

One way to generate synthetic genomics data is to use the SLIM open-source tool. As would be appreciated, SLIM is a genetically explicit forward simulation software package for population genetics and evolutionary biology. It is highly flexible, with a built-in scripting language, and has a cross-platform graphical modeling environment called SLiMgui. Other methods to generate synthetic genomics data are emerging, and can be expected to be introduced in the coming years.

In a synthetically generated subpopulation reference dataset it could be expected that subsequent generations may include direct descendants of the initial data elements that are used to seed the training models. To decrease bias resulting from the inclusion of the initial samples in the reference sequences, a process to remove the best matching haplotypes from the entire training set prior to use thereof in the population reference database can be employed. The greedy matching algorithm disclosed herein can be adapted for this purpose to better ensure the viability of a synthetically generated subpopulation reference dataset.

In regard to the decreasing of bias when generating synthetic datasets, when direct descendants are present in a training set, any matches between a specific sequence and related individuals as present in one or more reference population datasets can be removed. The inventors have determined that such matches can, in some implementations, result in the deep learning model will be trained on exact matches that can be expected to never happen in real life data instances. Removal of any exact matches when generating the synthetic dataset would cause the deep learning model to overfit on the exact matches to thus reduce the accuracy of the model when evaluating new test/sample DNA sequences. When a training option is enabled, the best subsequence match obtained during a scanning along the sequence in question in the recombination algorithm can be rejected as this best match can be associated with multiple matching lengths from multiple individuals in the reference sets. Removal of such best matches would remove matches from the given individual to one or both parental sequences.

Alternatively, or in addition to the removal of exact matches when training the deep learning model, a 3-way split of the data can be provided, such that the reference, test, training, and test can be separately accounted for when generating the synthetic datasets. A significant benefit of the present methodology when generating synthetic genomic datasets having utility in deriving local ancestry information for a person in need thereof is that the synthetic datasets herein are premised on recombination which is, of course, the biological basis from which real genomic data is generated in the first order. Thus, the inventors understand that the synthetic genomic data generated hereunder presents an improvement over prior art methods, such as those disclosed in US Patent Publication No. US2023/0326542 (the “'542 Publication), the disclosure of which is incorporated herein in its entirety by this reference. In one notable difference from the disclosure of the '542 Publication, the methodology therein is not configured to allow local ancestry information to be derived from a sample of genomic information having at least partially unknown and/or unconfirmed ancestry information.

Still further, the LAI determination engine can be configured with population genomic information derived from the actual genomic data and synthetic genomic data. Such a blended population reference database can be subjected to quality control steps, such as one or more filtering steps discussed previously.

Yet further, the population reference database can be evaluated from time to time to confirm the continued viability of the overall dataset, as well as the included subpopulation reference dataset for generation of accurate LAI information for a target individual. Such periodic testing and confirmation of the characteristics and context of the various subpopulation datasets used in the LAI information generation can assist in ensuring that LAI information generated therefrom continues to be accurate with regard to a target individual in need of LAI information therefrom. As needed, one or more subpopulation reference dataset can be filtered, cleaned, etc. to remove invalid data or datasets incorporating invalid data.

As would be appreciated, it can be expected that as genomics science continues to advance at a rapid rate, additional datasets including useful genomic data for individuals associated with one or more local ancestries will be generated. Such additional datasets can be combined with an existing population reference database both in terms of adding data points to existing reference populations and adding new reference populations to enhance the utility thereof in generating LAI determinations for use as set forth herein.

Yet further, one of ordinary skill in the art may wish to substitute newer datasets for previous datasets that may later be found to incorporate less reliable data. From time to time, additional subpopulation reference datasets can be added to the population reference database as more datasets become updated. Such updated datasets can comprise real-world genomics data, synthetic genomics datasets, or a combination thereof. In this regard, the population reference database can be seen as evolving over time to allow the use of data collection improvements that may be later developed.

The generated population reference database can have utility in generation of LAI information for an individual in need thereof—that is, a target individual. In this regard, genomic information for the first individual having an unknown or unconfirmed local ancestry can be provided for analysis in the LAI determination engine vis a vis the categorized genomic information in the population reference database. As described previously, subpopulation data elements in the population reference database comprises genomic information configured to allow LAI information to be derived from the first individual's genomic data. In some cases, more than one LAI determination can be generated for a target individual, for example, for a person who comprises an admixed individual.

As indicated, LAI information can be generated according to the methodology herein to provide enhanced accuracy over prior art LAI determination methods. This, in turn, can be expected to enhance generation of distinct local ancestry determinations between sub-populations that may otherwise be considered to be “closely related.” For example, not only can cross-continental ancestry determinations (e.g., Africa vs. Europe, South America vs, North America) be provided, sub-continental determinations (Mexico vs. Central America, Eastern Africa vs. Western Africa) can be generated with a high degree of accuracy. Moreover, such improved output can be used to extract useful information, as set out further hereinafter.

A LAI determination generated for a target individual can be provided with a level of accuracy of the information. For example, when a target individual is provided with a LAI determination, the system can also generate a probability that the provided information is accurate. In some implementations, a LAI generated for a target individual can be rejected or flagged if a probability of accuracy is below a threshold level, for example, having less than an about 90%, 85%, 80%, 75%, or 70% probability of being accurate, where accuracy is based on an actual local ancestry origin(s) for a target individual. When used in a medical or health-related application, the correctness (or lack thereof) of a generated LAI determination can be relevant. As such, it can be useful to provide any generated LAI for a target individual with information about accuracy.

In an implementation, the disclosure also provides methodology for analyzing a target individual's genomic information to generate or infer a local ancestry for the person via the LAI determination engine. Such information can have utility for use in various genomics consumer products, such as those provided by Ancestry.com, 23andme, among others. Today, these products provide limited information about the specific geographic ancestral origins of some individuals. For example, there is currently limited information about the local origins of persons whose ancestral origins are outside of Europe, especially persons whose ancestors may have been subjected to forced, coerced, or even voluntary migration of diasporic populations occurring in the past. The methodology herein can be useful to provide these individuals with enhanced information about the geographic origin(s) of their ancestors, even where documented family history may be limited or even non-existent. While such ancestry-related consumer products are often used to satisfy the curiosity of users who wish to “find their roots,” the information derivable therefrom can also have utility in identifying close relatives who might have or have a need for medical or health-related information having relevance to the target individual.

As would be appreciated, SNPs represent the most common type of genetic variation among humans. SNPs occur normally throughout a person's DNA. They occur almost once in every 1,000 nucleotides on average, which means there are roughly 4 to 5 million SNPs in a person's genome. These variations occur in many individuals; to be classified as a SNP, a variant is found in at least 1 percent of the population. Most commonly, SNPs are found in the DNA between genes. When SNPs occur within a gene or in a regulatory region near a gene, they may play a more direct role in disease by affecting the gene's function. While most SNPs exhibit no significant effect on an individual's medical condition or health, some of an individual's genetic differences can impart significant effects on a human's medical condition or health. SNP identification in genomic information thus has relevance in LAI, as well as in generating information about the susceptibility (or lack thereof) to one or more medical or health-related conditions. For example, the presence of certain—or target—SNPs can help predict an individual's response to certain drugs, susceptibility to environmental factors such as toxins, and risk of developing diseases.

As would be understood, persons of various ancestral origins can exhibit greater or lesser susceptibility to certain medical or health-related conditions. While a number of SNPs are today known to cause diseases as determined by existing genomic information analyses, as noted, such data can be biased toward the content of the databases, namely, European origin genomic data and, more recently, that of Asian origin. By allowing a LAI information to be cross tabulated along with the presence or absence of one or more SNPs or SNP groupings, improvements in medical or health-related condition prediction accuracy for an individual having association with one or more LAI determinations can be generated. In this regard, by aligning the presence or absence of one or more SNPs and/or SNP groupings with a LAI(s) determination for an individual, it may be possible to track the inheritance of disease-associated genetic variants as a function of the geographic origin(s) for that individual.

In various implementations, a number of SNPs may be of interest for detection in a target individual. For example, there can be at least about 10, 50, 100, 150, 200, 250, 300, 350, 400, 450, 500, 550, 600, 650, 700, 750, 800, 850, 900, 950, 1000 or more SNPs that can be of interest for detection in a target individual's genomic information, where such detected SNPs can be associated with one or more LAI determinations for a target individual based on information derivable from the population reference database. As would be appreciated, as genomic databases continue to expand and analyses thereof become more robust, additional SNPs or groupings of SNPs could be expected to become relevant for detection in relation to a susceptibility of an individual to one or more medical or health-related conditions. As such, the methodology herein is contemplated to have utility in such after-developed SNP identification and detection scenarios.

In a specific type of prediction or risk assessment relevant to the methodology herein, a target individual can be provided with one or more Polygenic Risk Scores (“PRSs”). As would be understood, a PRS is an estimate of an individual's genetic risk for presenting with or acquiring a medical or health-related condition. A PRS can be obtained by aggregating and quantifying the effect of many common variants (usually defined as minor allele frequency ≥1%) in an individual's genome, each of which can have a small effect on the person's genetic risk for a given disease or condition. A PRS is typically constructed as the weighted sum of a collection of genetic variants, usually SNPs defined as single base-pair variations from the reference genome. The resulting score is approximately normally distributed in the general population, with higher scores indicating higher risk of acquiring or presenting with a given disease or condition.

Of course, in order to be useful, a generated PRS score for an individual should be generated using reference genomic data that is relevant for that individual. As discussed previously, local ancestry is increasingly recognized as a preliminary consideration in the reliability of a PRS generated for an individual, not least because local ancestry can be a key determinant in the susceptibility of an individual to one or more medical or health-related conditions. Thus, it can be highly relevant to deconvolute the local ancestry aspects of an individual's genomic information prior to generating a PRS for that individual which, as would be seen by the disclosure hereinabove, can be provided by the novel methodology herein.

Yet further, the LAI determination engine can be used in generating LAI information for a target individual for whom determination of a local ancestral origin can be useful in diagnosing or predicting medical or health-related uses, for example, in generating a PRS associated with one or more medical or health-related conditions. As would be appreciated, a PRS can provide an individual with an assessment of the risk of the occurrence of or susceptibility to a specific condition based on the collective influence of many genetic variants. A PRS can be associated with recognized variants associated with genes of known function and variants not known to be associated with genes relevant to the condition. Alignment of genomic variation data with medical or health-related information from individuals who also have local ancestry that is accurately represented in the population reference database can enhance the reliability of a PRS generated for a target individual. Accordingly, use of relevant electronic medical records for a target individual can have utility when making a prediction of whether (or not) they may likely or probably present with or acquire one or more medical or health-related conditions that to which persons having the same or similar LAI determination also have a higher probability of being symptomatic for, as shown by analysis of one or more subpopulation reference datasets versus a comparison with the overall population reference database information. Such probabilities can be shown for one or more medical or health-related conditions in the form of one or a collection of PRSs generated for the target individual.

For example, if the target individual is a man of Western African local origin, his genomic information can be analyzed according to the methodology herein to identify the prevalence of (or lack thereof) of one or more medical or health-related conditions by analyzing his genomic information against that of other males determined to have his same or similar LAI determination. The analysis can provide the target individual, here the man of Western African origin, with a prediction of whether he is more or less likely to be symptomatic of or being diagnosed with one or more medical or health-related conditions.

Generated PRS or other forms of predictions for a target individual can include additional data elements such as age, economic status, education level, family status, birth location, home location, work location, etc. When available in the relevant datasets, such additional information can allow detection or identification of external factors that may increase or decrease a target individual's susceptibility to one or more medical or health-related conditions.

With regard to identification of a susceptibility of a target individual to presenting with or acquiring one or more medical or health-related conditions, such as can be provided by PRSs, medical histories (e.g., via electronic medical records) of individuals for whom genomic information is present in the population reference database can be associated with the subpopulation local ancestry information therein. Medical histories for a collection of non-admixed individuals having LAI determinations associated therewith can be compared to identify more or less propensity for being diagnosed with a medical or health-related condition. For example, if a collection of medical histories for a plurality of non-admixed individuals demonstrates that persons having a first LAI determination are more likely than other collections of persons having different LAI determinations to be diagnosed with a specific medical or health-related condition, the persons having the first LAI determination can be identified as being more likely to present with or acquire that specific condition. When a target individual is determined to be associated with the first LAI determination, that person can be provided with information, such in the form of a report, that indicates that the person may present with or acquire the specific condition for which persons in their LAI group have a greater susceptibility to, as shown by the collection of LAI information having medical record information associated therewith. For example, the target individual can be provided with PRS information that takes into consideration the LAI information for that person, such that a provided PRS is adjusted higher or lower with regard to one or more medical or health-related conditions.

PRSs can provide useful information for personalized risk stratification and disease risk assessment, especially when combined with non-genetic risk factors. To date, PRSs have been studied heavily in obesity, coronary artery disease, diabetes, breast cancer, prostate cancer, Alzheimer's disease and psychiatric diseases. As more expansive and accurate genomic data is generated from individuals, it can be expected that the utility of PRSs will be enhanced. To this end, PRSs could become a cost-effective way to generate information having value in clinical management of specific individuals or in a group of individuals presenting with collections of genomic characteristics (i.e., SNPs), that can be indicators of a potential or actual susceptibility to one or more medical or health-related conditions. Moreover, a PRS may be used during an individual's lifespan helping to quantify the genetic lifelong risk for one or more medical or health-related conditions. To this end, for some diseases, having a strong genetic risk can result in an earlier onset of presentation than might be seen in an individual not having those same genomic characteristics. Identification of a propensity or susceptibility to a disease or condition via PRS generation can allow clinical intervention earlier and, in some cases, can ameliorate many of the negative effects that might occur with a delayed diagnosis. PRS can be combined with traditional risk factors (e.g., obesity, smoking, etc.) to increase clinical utility. PRS generation may also motivate individuals to modify their lifestyles to reduce the risk of acquiring a medical or health-related condition for which they may be at risk for presenting with later in life as indicated by a generated PRS. Population level screening is another use case for PRS. The goal of population-level screening is to identify patients at high risk for a disease who would benefit from an existing treatment; the need therefore could be identified for the individual by such screening.

Information configured as a library of medical or health-related condition susceptibility for an individual having a LAI determination(s) along with actual diagnosis, treatment, and outcome information can be generated. The library can be configured for use in generating diagnosis and treatment recommendations for presentation to a medical provider. Information generated for a target individual can be added to the library to allow further expansion of the population thereof with useful data.

For some subpopulation reference datasets, medical information may not be natively associated with provided genomic data from which LAI information can be derived therefrom, such as is present in the UKBB. However, it is expected that more robust genomic data that is aligned with medical records will be available for use by researchers and commercial entities as genomic data sources and processing thereof become more ubiquitous. Some US health systems are collecting genomic data from patients, and such information will natively be associated with a patient's medical record as held by their medical provider. For example, the Million Veteran's Program (“MVP”) is a national research program looking at how genes, lifestyle, military experiences, and exposures affect health and wellness in Veterans. Since launching in 2011, more than 930,000 Veterans have joined MVP. This program aims to improve health care for US military veterans as one of the largest research programs in the world studying genes and health. The US National Institutes of Health is also acquiring genomic and health information from a large number of people in the “All of Us” research study. The All of Us Research Program is part of an effort to advance individualized health care by enrolling one million or more participants to contribute their health data over many years. The program aims to reflect the diversity of the United States and to include participants from groups that have been underrepresented in health research in the past. For some All of Us study participants, genomic and health information can be aligned with an individual's medical records.

Using appropriate privacy protections, health records associated with patient genomic data generated from such-large scale research programs can be provided for LAI determination and associated medical and health-related predictions etc. As discussed previously, such patient records can be masked or transformed to allow privacy to be maintained, while still enabling robust insights for individuals and groups of individuals to be derived therefrom.

In further implementations, the methodology herein can have utility in generating genomic information for a subpopulation of individuals having substantially the same ancestral geographic local origin. By categorizing a group of individuals' genomic data according to their genetic origin(s), it can be expected that improvements in assessing a susceptibility of that group to one or more medical or health-related conditions can be provided. As would be appreciated, alignment of medical records with genomic data associated with local ancestry could allow improvements in predicting the susceptibility of a group of individuals having the same or similar local ancestry to one or more medical or health-related conditions. As with predictions made for a single target individual, predictions made for a collection of target individuals can incorporate additional data elements that may affect or be relevant to such a prediction. Such additional data elements can include age, sex, economic status, education level, family status, birth location, home location, work location, etc.

In various implementations, the subpopulation reference datasets incorporated in the population reference database configured for use in the LAI determination engine can be configured for analysis of one or a plurality of haplotypes (collectively “haplotype configurations”) associated with one or more medical or health-related conditions of interest. As would be appreciated, in some cases, relevant haplotypes could be different between individuals having different local ancestries. In this regard, a target individual having a first haplotype configuration in their genomic information can be associated with a different susceptibility to one or more medical or health-related conditions than a second individual having a second haplotype configuration in their genomic information, where the differences in susceptibility can be attributed, at least in part, to a local ancestry that can be determined to be different between each of the first and second individuals. In this regard, different PRSs or other types of predictive products for a medical or health-related condition of interest for prediction can be generated for each of the first and second individuals.

The methodology herein can have utility in classifying genomic windows of one or more haplotype configurations in a target individual's genomic information, where the one or more haplotype configurations can be associated with genetic variations that might be relevant to their susceptibility to one or more medical or health-related conditions. Thus, the presence or absence of one or more haplotype configurations in an individual's genomic information can have relevance in generating medical or health-related information for an individual in need thereof.

In some implementations, the methodology herein has utility in the determination of genome-wide local ancestry in admixed individuals—that is, persons of mixed ancestry—from genomic information datasets, for example data in the UKBB. The methodology can provide association analyses of individuals having data incorporated therein at regional levels (e.g., sub-continental areas) to allow useful local origin information to be generated. For example, more granular information about an admixed individual's various local ancestry can be generated, such as by identifying two or more local ancestries for that person. This, in turn, can provide enhancements in risk prediction for that individual's susceptibility to one or more medical or health-related conditions.

Further, the methodology herein can provide utility in generating PRSs or other types of predictions associated with medical or health-related conditions for admixed individuals, that is, for individuals having genetic origins derived from at least two different local ancestries. These at least two different local ancestries can be generated using the methodology herein. In view of the increasing number of multi-ethnic unions in the modern world, the determinations can be expected to help address disparities in human health care delivery.

The methodology herein can also provide improvements in pharmacogenomics. Pharmacogenomics is the study of how genes affect a person's response to drugs. This field combines pharmacology and genomics to develop effective, safe medications that can be prescribed based on a target individual's specific genomic information. Currently, there is a lack of knowledge about how specific drugs and/or dosages affect individuals; instead, many drugs currently available are prescribed substantially as “one size fits all.” The methodology herein can be used to predict who will benefit from a medication, who will not respond at all, and who will experience an adverse drug reaction. Yet further, dosage information can be derived for a target individual. As would be appreciated, the combination of an accurate local ancestry determination for a target individual in comparison to others having the same or similar local ancestry can be analyzed along with medical records associated with the population reference database and the incorporated subpopulation reference datasets. Such information can be used to assist in selection of one or more medications for treatment of a medical or health-related condition in the first order.

The methodology herein also has utility for generation of medical screening policies where risk assessment can be adjusted for likely genetic and environmental effects without first having to subject the target individual to substantial testing or study. If a target individual's determined LAI determination indicates that they have a high probability of being diagnosed with one or more medical or health-related conditions currently or sometime in the future, they can be entered into a treatment or monitoring program without the need for, or at least the need for lesser, testing etc. Such detection can reduce the time, cost, and discomfort often associated with identifying an individual as needing monitoring and/or treatment for a medical or health-related condition. Persons can also be disqualified from a treatment etc. if it can be determined that a particular treatment is likely not to be effective for that person.

The methodology herein also can be expected to have utility in personalized medicine. Personalized medicine is an emerging practice of medicine that uses an individual's genetic profile to guide decisions made in regard to the prevention, diagnosis, and treatment of disease. Knowledge of a patient's genetic profile can help doctors select the proper medication or therapy and administer it using the proper dose or regimen. Since a person's local ancestry can affect their susceptibility to a medical or health-related condition, determination thereof according to the disclosed methodology can be expected to improve personalized medicine methodologies.

The methodology herein can have utility in drug discovery for specific treatments targeted according to local origin of an individual or collection of individuals. The processes herein can allow identification of test subjects according to local ancestry origin determination. The genomic information for such individuals can be used to target drug discovery, such as by detecting genetic causes or drivers of one or more disease states. By centering drug discovery at least in part according to a person's local ancestry origin, it can be expected that medicine can become better targeted.

The LAI determination methodology can also aid in the development of new antibiotics and antimycotics. As noted previously, ancestral contact with various microbes is known to have some effect on the susceptibility (or lack thereof) of some individuals to infections etc. caused by such microbes. Moreover, the effectiveness of treatments can also be affected by local ancestries, in some cases. It follows, selection of optimum antibiotics and antimycotics can also be improved with use of local ancestry information for an individual in need of treatment.

Research is emerging demonstrating that geographic location of origin can directionally influence the nature and character of a person's gut microbiome. Variations in the skin microbiome have also been observed. By generating information associated with a person's local ancestry origin, selection of treatments directed toward addressing a person's gut or skin microbiome, be it in the form of medication, nutraceutical, or cosmetic treatments, can be enhanced.

Cosmetic product design and selection can also be enhanced with use of the LAI determination methodology herein. An individual could be provided with their local origin information to assist in the selection of cosmetic treatments that are specifically configured for their skin or hair type. In this regard, ethnicity is known to be a key genetic trait when it comes to skin structure, as racial differences lead to variation in skin physiology. The composition of the stratum corneum and dermis, and the elasticity, collagen, and pigment levels of our skin, are all examples of physiological trains that can differ across different racial groups. For instance, structural and functional differences have been found in the skin of African American, Caucasian and East Asian populations. Within various East Asian subpopulations, significant differences were seen in skin characteristics. Further, differences in odor-causing excretions from the sebaceous glands (e.g., in axillary and pubic regions) have been observed depending on the local ancestral origin of individuals, a fact which can alter the effectiveness of cosmetic treatments. Skin microbiome variations, such as lower abundances of Staphylococcus spp. bacteria and greater abundances of Corynebacterium spp., have also been linked to physical differences that are related to different genotypes in people from East Asia and Europe or Africa when looking at the axillary microbiota.

The methodology herein also has utility in the design and evaluation of medical studies. In this regard, by adding an additional data element to each member of a study cohort, data analysis and associated information generation can be expected to provide more reliable results. For example, addition of an accurate LAI determination for members of a study cohort could at least reduce, or maybe even eliminate, confounding issues generated in study data. Yet further, selection of study participants at least in part based on LAI determination can assist in the tailoring of clinical trials to the more granular trait of local ancestry. A clinical trial can then be conducted to determine whether a drug or other treatment being tested therein is effective (or not) on an individual having a LAI determination associated with a specific geographic location, region etc.

In a further implementation, the LAI determination methodology can have utility in cancer diagnosis and treatment by allowing researchers to determine if ancestral background affects the types of mutations seen in various cancers and if this allows better diagnosis and prognostication in addition to more customized therapy targeting these cancers. For example, local ancestry origin determinations as derived from the methodology herein can be used to select subjects from which biological samples can be provided so as to test the effectiveness of new drug candidates to include an additional variable of local ancestry. The results from such treatment effect determinations can be used to select and/or deselect patients from treatment with various oncology drugs/immunotherapies in accordance with an amount of effectiveness that such treatment was shown to demonstrate as a function of a patient's local ancestry origin.

The LAI information generated herein can also have utility for use in enhancement of other types of information generation. For example, the information can be used to improve existing PRS scores, such as by enabling validation of existing scores and, if appropriate, modifying these determinations when appropriate. The validity or reliability of a PRS score generated for a target individual or group of individuals can be tested and, if appropriate, information used to generate the PRS can be modified if it is found that the PRS was not, in fact, accurate to identify a susceptibility of the individual to present with or acquire a medical or health-related condition.

The LAI determination engine and information generated therefrom can be used to evaluate the different effects (or lack thereof) of a genetic variant in different subpopulations. For example, a variant known to cause a first disease state can be examined according to a local ancestry determination for a collection of medical data associated with a collection of individuals to determine how that variant does (or does not) increase a susceptibility to a medical or health-related condition known to be associated with that variant. Such information can be used to improve the quality of PRSs determinations for persons having specific local origin(s).

The methodology herein also can have utility in GWAS, such as by enhancing the signal strength for each region based on a local ancestry determination. Discovery of ancestral origins associated with different sections of a person's DNA can be used in GWAS to identify the actual variants driving disease, what is sometimes referred to as fine mapping. When testing for an association between a variant and a disease, there are multiple things that can confound the results. Additionally, because of linkage disequilibrium, it can be hard to tell which variants may be only associated with a disease from variants that are actually causal in driving a medical or health-related condition presentation now or in the future. By including local ancestry information, causal variant information can be disaggregated, and it can be expected that such information can enhance drug discovery and design, as well as improving diagnosis and associated outcomes.

Genotype imputation is a method for inferring missing genotypes given some set of observed or measured genotypes. It takes advantage of linkage disequilibrium and recombination in addition to statistical models to infer more genotypes than were measured. It is a common and widely used technique. By adding local ancestry information this technique can be improved, thereby allowing use thereof in a clinical setting. The inventors hypothesize that clinical use of genomic data can be available, at least because expensive sequencing techniques could be substituted, at least in part, with more focused genomic analysis associated with an individual's local ancestry information.

“Phasing” is the process of taking genotype or sequencing data and associating it with one of the two parental chromosomes. From such information, one can generate information associated with whether variants of interest have maternal and paternal origins. Current techniques have limitations and break down in phasing over long ranges, to often result in a chunk phased on the wrong chromosome. Local ancestry can be used to correct this current limitation with existing phasing methods to more precisely put the variants together on the maternal and paternal chromosomes. This can have utility in applications spanning a number of fields as this is an ongoing issue with current techniques used to determine individuals' variants.

The methodology can also have utility in correction of population structure for datasets that are used in genomic studies. In this regard, PCA is a technique routinely used to adjust for global allele frequency differences between subpopulations in genetic association studies. However, PCA does not account for local ancestry at any specific locus. The disclosed method can be used to substitute for or to augment PCA to enhance the accuracy of the underlying datasets before using the data therein to generate predictions. Such fine scale improvements can enhance the utility of data generated from genomic analysis.

Use of the accurate LAI determination processes can also be used in reducing data dimensionality, as a Gaussian filter for genomics studies, and to make more discrete data classifications. Again, such fine scale improvements can enhance the utility of data generated from genomic analysis.

The LAI determinations that are possible with the methodology can also allow unusual or uncommon genetic samples to be reduced to dimensionally simpler samples that fit into more fundamental data classification structures. In other words, local ancestry deconvolution enabled according to the methodology herein could be expected to allow some individual genomic data to be simplified so as to allow it to be confidently incorporated into a subpopulation reference dataset for use in a population reference database.

The methodology herein can also be used in ancestry abstraction. This is a way of classifying regions of the genome by how closely related they are to a reference panel.

LAI determinations as disclosed herein can also be used in a transformer model as a positional embedding to pass ancestry structure information to enable the transformer to train better/faster for downstream tasks. In an implementation, the LAI determination information itself can be a form of dimensionality reduction that can allow these very large transformer models to better learn how to perform a task on genetic data. In some aspects, a dimensionality reduction technique specific to genomic data and these large language models can serve either as dimensionally reduced annotation data providing contextual information or input data itself.

The methodology herein further can have utility in improving synthetic genomic datasets. As set out above, synthetic datasets are becoming widely used in genomics products today. Using the local ancestry insights derivable using the methodology herein, synthetic datasets can be enhanced to incorporate more robust information associated with local ancestry, thus increasing not only the accuracy of such synthetic datasets, but also their utility. In this regard, an output or any associated data from one or more LAI determinations can be input into subsequent local ancestry determination processes for generation of local ancestry determinations for one or more individuals.

The methodology herein can also have utility in the examination of gene by gene interactions and epistatic effects due to improved phasing. Genetic interactions can be characterized by two or more variants producing an unexpected phenotype that is not easily explained by the marginal effects of the individual variants. In other words, the same mutation can have different effects in different individuals. One reason for this is that the outcome of a mutation can depend on the genetic context in which it occurs. The local ancestry deconvolution available according to the processes herein can be expected to enable the reasons for some currently unresolved epistatic effects to be generated from evaluation of genomic information derived from a plurality of subpopulation reference datasets. Yet further, the processes herein can also provide insights into how genetic admixtures may influence, or not, epistatic gene networks. Information derivable therefrom has utility in generating genomic information datasets and in uses thereof.

In a further aspect, the methodology herein can be used to better understand gene by environment interaction. In this regard, when a collection of subpopulation reference dataset information is determined to comprise the same or similar LAI determination information, local ancestry can be used as a proxy for socioeconomic/environmental factors. Once an individual's ancestral origins of one or more variants can be determined, such information can have utility in other useful determinations. For instance, a target individual's genomic data can be analyzed to determine whether a variant present in their genomic data may be associated with specific medical or health-related condition (e.g., heart disease, diabetes, etc). If the association vanishes in another individual having the same or similar LAI determination, then it could be concluded that the presence of the subject variant in the target individual's genomic data could comprise confounding information or may be an interaction between some environmental variable and the variant.

Gene expression quantitative trait loci (eQTL) can provide mechanistic insights for genetic variants associated with complex traits in GWAS. In some aspects, generation of a highly accurate local ancestry adjustment could improve statistical power in both cis and trans-eQTL mapping. Moving from DNA variants to expression of RNA could have utility for finding mechanisms by usefulness as drug targets in drug discovery processes. Accurate mapping of ancestry at the local level; this information could allow improved determination of causal variants that change the expression of RNA. In turn, this could allow better designs for drugs to target these changes.

Also, in GWAS-eQTL colocalization at subcontinental level a GTEx dataset could be re-analyzed in the context of LAI determinations since the information would include more admixed African-American individuals and white participants who may have different European ancestry backgrounds.

In addition to being useful in human genomics, the LAI determination methodology herein can be extended to animals to, for example, guide breeding. Accurate ancestry for animals to detect breeding/migration patterns can also be determined.

In plants, the methodology can also be used to guide selection of characteristics that might be desirable in crops. For example, LAI determination could be used as an alternative to GWAS to isolate the effect of different genes in crops and maximize desired traits. Crop detection, such as for proprietary characteristic determination, could utilize LAI determination in place of, or to augment, genetic marker tracing. For example, seed companies could detect if any of their registered hybrids/varieties have been used to breed an unauthorized offspring.

LAI determinations can have use as a context to feed to machine learning models (many different kinds: LSTM, transformer, convolutional) to act as a modifying factor of the activity of a site. This could be used for many distinct types of ML models for PRS. In this regard, LAI determinations can be input in AI/ML models to improve downstream predictions based on DNA data. As an example, prediction of a cancer risk or the type of gene that may be associated with drug side effects or lack of efficacy can be enhanced by feeding the ancestry information along with the DNA sequences. AI/ML models could be expected to make better predictions.

In a transformer model it can be possible to use an “ancestry embedding” to modify the input tokens to make those of similar ancestry more similar in terms of sequence input to a downstream model.

In yet another implementation, the methodology herein can be used in conjunction with CRISPR gene editing induced large structural changes at both on target and off-site locations to better understand the long term evolutionary consequences of this emerging technology outside of the laboratory setting.

Those skilled in the art will recognize that one or more of the steps in the methodologies set forth herein are performed on a computing device. Therefore, in some implementations, it is expressly contemplated that one or more of the steps disclosed herein are implemented on a computing device. A computing device refers to a functional unit that contains one or more standalone or interconnected processors operably linked to a tangible, non-transitory computer-readable media. “Operably linked” refers to the connection of at least two components in the computing device via technology including, but not limited to, integrated circuits, internet, ethernet, intranet, Bluetooth, near field communication, WiFi, or a combination thereof. Computing devices include, but are not limited to, desktop computers, laptop computers, tablet computers, servers, and mainframes.

APPENDIX I

| Data source | Description | Genotyping technology | Populations |
|---|---|---|---|
| Byrska-Bishop | 1000 Genomes Project | Whole genome sequencing | CSA, GSE, GLS, NGA, |
| et al, 2022 | | | CHD, CHI, VIE, JPK, |
| Lowy-Gallego | | | FIN, ITA, SPP, BNI, |
| et al, 2019 | | | GUP, NIP, ISL |
| Bergström et al, | Human Genome | Whole genome sequencing | CSA, GSE, NGA, CHI, |
| 2020 | Diversity Project | | SEA, JPK, MAM, MEL, |
| | | | ITA, SPP, ARB, LEV, |
| | | | NAF, CEA, NIP |
| Mallick et al, | Simons Genome | Whole genome sequencing | CSA, GSE, NGA, FIL, |
| 2016 | Diversity Project | | SIB, MEL, EAE, ITA, |
| | | | SPP, ARB, LEV, NAF, |
| | | | ICM, CEA |
| To be published | Artificially | Whole genome sequencing | NAM |
| by inventors | constructed Native | (from 1KGP, HGDP and SGDP | |
| | Americans | genomes) | |
| Almarri et al, | Middle Eastern | Whole genome sequencing | ARB |
| 2021 | populations | | |
| Malaria | Gambian Genome | Whole genome sequencing | GSE |
| Genomic | Variation Project | | |
| Epidemiology | (Fula, Jola, Mandinka | | |
| Network, 2019 | and Wolof ethnic | | |
| | groups) | | |
| Zhang et al, | Korean Personal | Whole genome sequencing | JPK |
| 2014 Kim et al, | Genome Project | (Illumina Hiseq) | |
| 2018 | | | |
| Carmi et al, | The Ashkenazi | Whole genome sequencing | ASK |
| 2014 | Genome Consortium | | |
| | (healthy individuals of | | |
| | Ashkenazi Jewish | | |
| | descent) | | |
| Bycroft et al, | UK Biobank project | UK BILEVE Axiom and UK | CSA, NEA, GSE, GLS, |
| 2018 | (participants from | Biobank Axiom arrays | NGA, CHI, FIL, SEA, |
| | across the United | (imputation with the Haplotype | VIE, JPK, MAM, EAE, |
| | Kingdom) | Reference Consortium, UK10K | BRI, FIN, FRG, SCA, |
| | | and 1000 Genomes reference | GBA, ITA, SPP, LEV, |
| | | panels) | NAF, CYP, ICM, CEA, |
| | | | NEP, BNI, NIP, ISL |
| Wang et al, | Modern samples from | Affymetrix Human Origins | CSA, NEA, CHI, FIL, |
| 2021 | the ‘1240K + HO’ | array | MAM, SIB, ASK, EAE, |
| Jeong et al, | dataset provided by Dr | | GBA, SPP, ARB, LEV, |
| 2019 | David Reich | | NAF, ICM, CEA |
| Biagini et al, | laboratory | | |
| 2019 | | | |
| Vyas et al, 2017 | | | |
| Skoglund et al, | | | |
| 2017 | | | |
| Skoglund et al, | | | |
| 2016 | | | |
| Lazaridis et al, | | | |
| 2016 | | | |
| Lazaridis et al, | | | |
| 2014 | | | |
| Pickrell et al, | | | |
| 2012 | | | |
| Anagnostou et | Berbers and Arabs | Illumina Human | NAF |
| al, 2020 | from Southern Tunisia | OmniExpressExome v 8.1 array | |
| Henn et al, | Berbers and Arabs | Affymetrix 6.0 array | LEV, NAF |
| 2012 Arauna et | from North Africa and | | |
| al, 2017 | Syria | | |
| Hollfelder et al. | Sudanese and South | Illumina Human | NEA |
| 2017 | Sudanese populations | Omni5MExome array | |
| Dobon et al, | Populations (Arabs, | Illumina Infinium Immunochip | NEA |
| 2015 | Beja, Ethiopian and | | |
| | Nubian) from | | |
| | Sudanese region | | |
| Behar et al, | Ashkenazi Jews and | Illumina Human610- | NEA, ASK, EAE, ICM |
| 2013 | non-Ashkenazi | Quad, Human660W-Quad, | |
| | populations from | HumanOmniExpress-12v1 | |
| | Eastern Europe, | 730K and HumanOmni1-Quad | |
| | Northeast Africa and | array | |
| | the Caucasus | | |
| Yunusbayev et | Caucasians and | Illumina 610K array | EAE, GBA, ICM, CEA |
| al, 2012 | geographically nearby | | |
| | populations (Central | | |
| | Asia, Eastern Europe | | |
| | and Balkans) | | |
| Yunusbayev et | Turkic-speaking | Illumina 550k, 610k, 650k and | MAM, EAE, ICM, CEA |
| al, 2015 | populations from | Human1M-Duo BeadChips | |
| | regions across Eurasia | | |
| | and their geographic | | |
| | neighbors | | |
| Behar et al, | Jewish Diaspora | Illumina Human610-Quad and | NEA, ASK, EAE, SPP, |
| 2010 | communities and non- | Human660W-Quad bead arrays | ARB, LEV, NAF, ICM, |
| | Jewish neighbor | | CEA |
| | populations from | | |
| | Europe, Asia and | | |
| | Africa | | |
| Tambets et al, | Uralic-speaking | Illumina Human610-Quad, | EAE, FIN |
| 2018 | populations and local | HumanHap650Y and | |
| | geographic neighbors | Human660W-Quad BeadChip | |
| Botigué et al, | Spanish from South | Affymetrix 6.0 array | SPP |
| 2013 | (Andalusian) and | | |
| | Northwest (Galician) | | |
| | Spain | | |
| Flores-Bello et | Basques (from France | Axiom Genome-Wide Human | SPP |
| al, 2021 | and Spain) and | Origins 1 Array | |
| | Spanish Peri-Basques | | |
| Henn et al, | Basques from Spanish | Affymetrix 6.0 array | SPP |
| 2012 | Basque country | | |
| Pathak et al, | Northwest Indian | Illumina HumanOmniExpress - | NIP |
| 2018 | populations from | 24 BeadChip | |
| | Rajasthan and | | |
| | Haryana states | | |
| | (Gujjar, Kamboj, Ror) | | |
| Nelson et al, | Indian Asians | Affymetrix GeneChip 500K | GUP, NIP |
| 2008 | (Gujarati, Hindi, | array | |
| | Punjabi, Pushto and | | |
| | Urdu) from the | | |
| | Population Reference | | |
| | Sample (POPRES) | | |
| | project | | |
| Changmai et al, | Khmer and Kuy from | Affymetrix Human Origins | SEA |
| 2022 | mainland Southeast | SNP array | |
| | Asia (Thailand) | | |
| Tatte et al, 2019 | Lao from Laos | Illumina OmniExpress | SEA |
| | | BeadChips for 650k, 710k and | |
| | | 730k | |
| Mörseburg et | Island Southeast | Illumina OmniExpress | FIL, SEA |
| al, 2016 | Asian populations | BeadChip for 730k | |
| | (Malay, Igorot, Luz) | | |

APPENDIX II
Ancestry classification for the 35 reference populations and associated codes.
Hierarchy is shown in three levels, corresponding to the continental (level
1), region-wide (level 2) and sub-regional population (level 3) scale.

| | L1 | | L2 | | L3 |
|---|---|---|---|---|---|
| Level 1 | code | Level 2 | code | Level 3 | code |
| Sub-Saharan African | SAF | West African | WAF | Nigerian | NGA |
| Sub-Saharan African | SAF | West African | WAF | Ghanaian, Liberian & | GLS |
| | | | | Sierra Leonean | |
| Sub-Saharan African | SAF | West African | WAF | Gambian & Senegal | GSE |
| Sub-Saharan African | SAF | Northern East African | NEA2 | Northern East African | NEA |
| Sub-Saharan African | SAF | Central, South & Southern | CSA2 | Congolese & Southern | CSA |
| | | East African | | East African | |
| Western Asian & | WAN | Arab & Levantine | ALE | Arab | ARB |
| North African | | | | | |
| Western Asian & | WAN | Arab & Levantine | ALE | Levantine | LEV |
| North African | | | | | |
| Western Asian & | WAN | North African | NAF2 | North African | NAF |
| North African | | | | | |
| Western Asian & | WAN | Northern West Asian | NWA | Iranian, Caucasian & | ICM |
| North African | | | | Mesopotamian | |
| Western Asian & | WAN | Northern West Asian | NWA | Cypriot | CYP |
| North African | | | | | |
| Central & South | CAS | Central Asian | CEA2 | Central Asian | CEA |
| Asian | | | | | |
| Central & South | CAS | Northern South Asian | NSA | Nepali | NEP |
| Asian | | | | | |
| Central & South | CAS | Northern South Asian | NSA | Northern Indian & | NIP |
| Asian | | | | Pakistani | |
| Central & South | CAS | Northern South Asian | NSA | Bengali & Northeast | BNI |
| Asian | | | | Indian | |
| Central & South | CAS | Northern South Asian | NSA | Gujarati Patidar | GUP |
| Asian | | | | | |
| Central & South | CAS | Southern South Asian | SSA | Southern Indian & Sri | ISL |
| Asian | | | | Lankan | |
| European | EUR | Northwest European | NWE | British & Irish | BRI |
| European | EUR | Northwest European | NWE | Scandinavian | SCA |
| European | EUR | Northwest European | NWE | Finnish | FIN |
| European | EUR | Northwest European | NWE | French & German | FRG |
| European | EUR | Eastern European | EAE2 | Eastern European | EAE |
| European | EUR | Southern European | SEU | Spain & Portugal | SPP |
| European | EUR | Southern European | SEU | Italian | ITA |
| European | EUR | Southern European | SEU | Greek & Balkan | GBA |
| European | EUR | Ashkenazi Jewish | ASK2 | Ashkenazi Jewish | ASK |
| East Asian | EAS | Chinese & Southeast | CSE | Vietnamese | VIE |
| | | Asian | | | |
| East Asian | EAS | Chinese & Southeast | CSE | Chinese | CHI |
| | | Asian | | | |
| East Asian | EAS | Chinese & Southeast | CSE | Chinese Dai | CHD |
| | | Asian | | | |
| East Asian | EAS | Chinese & Southeast | CSE | Filipino | FIL |
| | | Asian | | | |
| East Asian | EAS | Chinese & Southeast | CSE | Southeast Asian | SEA |
| | | Asian | | | |
| East Asian | EAS | Japanese & Korean | JPK2 | Japanese & Korean | JPK |
| East Asian | EAS | Northern Asian | NAS | Siberian | SIB |
| East Asian | EAS | Northern Asian | NAS | Manchurian & Mongolian | MAM |
| Native American | NAM1 | Native American | NAM2 | Native American | NAM |
| Melanesian | MEL1 | Melanesian | MEL2 | Melanesian | MEL |

What is claimed is:

1. A method for generating local ancestry inference (LAI) information for a target individual, the method comprising:
a. processing a target individual's genetic information with a LAI determination engine configured with deep learning capabilities, the processing comprising:
i. deriving base layer information output from the target individual's genomic information, wherein the base layer information includes a preliminary determination of a first degree of relatedness between the target individual and individuals having genomic information incorporated in a plurality of subpopulation reference datasets each associated with a subpopulation geographic category, wherein each subpopulation reference dataset comprises assignments of individual LAIs for each dataset as derived from processing of genomic information obtained from collections of non-admixed individuals, and wherein each assigned LAI is associated with a specific geographic region or location; and
ii. performing one or more smoothing operations on the base layer information output to derive a second degree of relatedness between the individuals having genomic information incorporated in each of the subpopulation reference datasets and the target individual;
b. generating, on a computing device, one or more LAI determinations for the target individual; and
c. configuring the one or more LAI determinations for use with medical or health-related information associated with the target individual.

2. The method of claim 1, comprising prior to step (a):
a1. providing the plurality of subpopulation reference datasets each associated with a subpopulation geographic category;
a2. configuring each of the subpopulation reference datasets for use in a population reference database; and
a3. providing genomic information for the target individual associated with a LAI that is at least partially unknown or unconfirmed.

3. The method of claim 1, wherein the LAI information generated for each reference subpopulation dataset is generated from analysis of a plurality of single nucleotide polymorphisms (SNPs) present in the genomic data derived from the collection of non-admixed individuals.

4. The method of claim 3, wherein the LAI determination engine is configured to:
ax. analyze a plurality of single nucleotide polymorphisms (SNPs) present in the target individual's genomic data for use in generating the one or more LAI determinations; and
bx. compare the output from the analysis of the target individual's SNPs with the LAI information generated from the analysis of the SNPs present in the genomic data derived from the collection of non-admixed individuals.

5. The method of claim 2, wherein each of the one or more LAI determinations for the target individual is associated with a probability assessment of a presence or absence of a minimum level of accuracy, thereby providing a collection of LAI determinations identified as suitable for subsequent use in the LAI determination engine.

6. The method of claim 5, wherein each of the LAI determinations identified as suitable for subsequent use are configured for use in training sets associated with the LAI determination engine.

7. The method of claim 2, wherein genomic data in each of the subpopulation reference datasets are analyzed for a degree of relatedness that indicates that individuals are related as parent and child, and if the analysis indicates that individuals are related as parent and child, the genomic data associated with such related individuals is removed from the subpopulation reference dataset prior to use in the reference population database.

8. The method of claim 2, wherein the LAI determination information associated with each of the subpopulation reference datasets is reviewed by a human prior to incorporation in the reference population database.

9. The method of claim 2, wherein the configuring of the subpopulation dataset comprises:
cx. determining whether one or more of the subpopulation datasets are in need of reconfiguration prior to incorporation in the population reference database; and
dx. performing a data reconfiguration for each of the subpopulation datasets determined to be in need of reconfiguration.

10. The method of claim 9, wherein the data reconfiguration for a subpopulation dataset determined to be in need of configuration comprises performing one or more data filtering steps on the dataset.

11. The method of claim 10, wherein the one or more data filtering steps comprise:
ex. selecting a minimum level of accuracy associated with a LAI determination generated for the target individual;
fx. analyzing each of the subpopulation datasets to identify data characteristics associated with generating a LAI determination for the target individual that is below the selected minimum level of accuracy; and
gx. performing one or more filtering operations on the subpopulation dataset to remove data associated with generation of a LAI determination that is below the selected minimum level of accuracy prior to use of the subpopulation data in the reference population database.

12. The method of claim 2, wherein more than one LAI determination is generated for the target individual.

13. The method of claim 12, wherein a probability that each of the LAI determinations for the target is accurate is provided for each of the generated LAI determinations.

14. The method of claim 2, wherein one or more of the subpopulation reference datasets is derived from synthetic data generated to simulate genomic data of a collection of non-admixed individuals, wherein the synthetic data includes simulated LAI information.

15. The method of claim 14, wherein prior to use of the generated synthetic subpopulation datasets in the reference population database the dataset is analyzed to remove genomic data associated with individuals that simulates a degree of relationship in the simulated data that indicates a relationship of parent and child.

16. The method of claim 2, further comprising updating the one or more LAI determinations for the target individual when additional reference subpopulation datasets are added to the reference population database.

17. The method of claim 2, wherein the specific geographic location or region comprises a continental or sub-continental region.

18. The method of claim 2, wherein the population reference database includes medical or health-related information for some or all of the individuals having genomic information in the subpopulation reference datasets, and the method further comprises:
hx. comparing the target individual's genomic information with the genomic information and medical- or health-related information for individuals in the population reference database having the same one or more LAI determinations as the target individual; and
ix. generating information associated with a probability that the target individual will present with or acquire one or more medical or health-related conditions as compared to other individuals having the same one or more LAI determinations.

19. The method of claim 2, wherein the generated or more LAI determinations for the target individual is used in generating one or more polygenic risk scores (PRS) for the individual.

20. The method of claim 2, wherein the LAI determination engine is configured to analyze a collection of haplotype configurations in the target individual associated with one or more medical- or health-related conditions, thereby providing information about a presence or absence of one or more genetic variations associated with a presence or absence of a medical- or health-related condition of interest in the target individual.
